(19) United States
(12) Patent Application Publication
(10) Pub. No.: US 2020/0364909 A1
(43) Pub. Date: Nov. 19, 2020

(54) SPECTRAL IMAGING USING A ROTATING SPECTRAL FILTER

(71) Applicant: KONINKLIJKE PHILIPS N.V., EINDHOVEN (NL)

(72) Inventors: CHUANYONG BAI, SOLON, OH (US); SHENG LU, HIGHLAND HEIGHTS, OH (US); HAO DANG, MAYFIELD HEIGHTS, OH (US); DOUGLAS B. MCKNIGHT, CHARDON, OH (US)

(21) Appl. No.: 15/733,122

(22) PCT Filed: Nov. 19, 2018
(86) PCT No.: PCT/EP2018/081668

§ 371 Date: May 21, 2020

Related U.S. Application Data
(60) Provisional application No. 62/591,315, filed on Nov. 28, 2017.

(51) Int. Cl. G06T 11/00; G01N 23/046; G01T 1/36

(52) U.S. Cl. CPC G06T 11/005; A61B 6/032; G01T 1/36; G01N 23/046

(57) ABSTRACT

An imaging system includes an X-ray tube (202) having a focal spot (204) and a port window (206), and a filter (208) having at least a first region (310) with a first material having first X-ray attenuation characteristics for a redetermined X-ray photon energy range of interest and a second region (312) with a different X-ray attenuation characteristic. The filter is disposed between the port window and an examination region (108) and is configured to rotate such that the at least the first and the second regions sweep through and filter X-ray radiation emitted from the focal spot. The system further includes an X-ray radiation flux detector (2802, 2902) configured to detect an X-ray radiation flux of the filtered X-ray radiation, a detector array (112) configured to detect the filtered X-ray radiation traversing the examination region and produce a signal indicative thereof, and a reconstructor (114) configured to process the signal based on the detected flux to reconstruct volumetric image data.

### FIELD OF THE INVENTION

The following generally relates to spectral (multi-energy) imaging and more particular to using a rotating spectral filter to filter the emitted X-ray radiation beam, and is described with particular application to computed tomography (CT) spectral imaging.

### BACKGROUND OF THE INVENTION

Computed tomography scanners configured for spectral imaging have used different approaches to obtain data at different energy spectra. One approach includes using multiple X-ray tubes, each emitting an X-ray beam having a particular energy spectrum, and corresponding multiple detector arrays. Unfortunately, this approach increases overall system cost relative to a scanner with a single X-ray tube and single detector array. Furthermore, X-ray radiation is ionizing radiation, which can damage and kill cells, and this approach may increase patient radiation dose relative to a single X-ray tube system. Another approach uses a dual layer detector in which a top layer detects lower energy X-ray photons and a bottom layer detects higher energy X-ray photons. This may lead to increased detector cost relative to a configuration with only a single detector layer. Another approach uses fast kVp switching. Generally, fast kVp switching for dual energy means the voltage across the tube is switched between two different voltages within each integration period such that two different energy measurements are taking each integration period. However, the sampling bandwidth is limited by the speed of the kVp switch, and there is a tradeoff between spatial resolution/image quality and temporal resolution. For example, for better temporal resolution, faster gantry rotation is required; however, due to the kVp switch speed limit, a smaller number of data is acquired at each rotation, negatively impacting the spatial resolution and image quality.

### SUMMARY OF THE INVENTION

Aspects described herein address the above-referenced problems and others.

In one aspect, an imaging system includes an X-ray tube having a focal spot and a port window, and a filter having at least a first region with a first material having first X-ray attenuation characteristics for a predetermined X-ray photon energy range of interest and a second region with a different X-ray attenuation characteristic. The filter is disposed between the port window and an examination region and is configured to rotate such that the at least the first and the second regions sweep through and filter X-ray radiation emitted from the focal spot. The system further includes an X-ray radiation flux detector configured to detect an X-ray radiation flux of the filtered X-ray radiation, a detector array configured to detect the filtered X-ray radiation traversing the examination region and produce a signal indicative thereof, and a reconstructor configured to process the signal based on the detected flux to reconstruct volumetric image data.

In another aspect, a method includes rotating a filter in a path of X-ray radiation emitted from an X-ray tube of an imaging system during scanning. The filter includes at least a first region with a first material having first X-ray attenuation characteristics for a predetermined X-ray photon energy range of interest and a second region with a different X-ray attenuation characteristic. The method further includes detecting a position of the filter based on an X-ray radiation flux, and reconstructing acquired data based on the detected flux to reconstruct volumetric image data of interest.

In another aspect, a computed tomography imaging system includes an X-ray tube and a filter with at least a first region with a first material having first X-ray attenuation characteristics for a predetermined X-ray photon energy range of interest and a second region with a different X-ray attenuation characteristic. The filter is cylindrically shaped, configured to surround the X-ray tube, and configured to rotate such that the at least first and the second regions sweep through and filter X-ray radiation emitted from the X-ray tube. The system further includes a detector array configured to detect the X-ray radiation traversing the filter and produce a signal indicative thereof, and a reconstructor configured to process the signal to reconstruct volumetric image data.

In another aspect, a computed tomography imaging system includes an X-ray tube, a plurality of moveable filters, each including a different first material having different X-ray attenuation characteristics and a second region with a second X-ray attenuation characteristic, and a drive system configured to move a predetermined one of the plurality of moveable filters into a path of X-ray radiation emitted from the X-ray tube. The system further includes a detector array configured to detect the X-ray radiation traversing the filter, and produce a signal indicative thereof and a reconstructor configured to process the signal to reconstruct volumetric image data.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an example CT imaging system with an X-ray sub-system configured for spectral imaging.

FIG. 2 schematically illustrates an example of the X-ray sub-system.

FIG. 3 schematically illustrates an example of an X-ray energy spectrum filter of the X-ray sub-system of FIG. 2.

FIG. 4 schematically illustrates an example of the filter of FIG. 3 in connection with an X-ray tube and a detector array in an x-y plane of the imaging system.

FIG. 5 schematically illustrates an example of the filter of FIG. 3 in connection with an X-ray tube in a z-y plane of the imaging system with the filter outside of the X-ray beam path.

FIG. 6 schematically illustrates an example of the filter of FIG. 3 in connection with an X-ray tube in the z-y plane of the imaging system with the filter completely inside of the X-ray beam path.

FIG. 7 schematically illustrates an example of the filter of FIG. 3 in connection with an X-ray tube in the z-y plane of the imaging system with the filter partially inside of the X-ray beam path.

FIG. 8 schematically illustrates a variation of the filter of FIG. 3 in which filter regions are on an interior of the filter support.

FIG. 9 schematically illustrates another variation of the filter of FIG. 3 in which filter regions span an exterior and an interior of the filter support.

FIG. 10 schematically illustrates yet another variation of the filter of FIG. 3 in which filter regions are within the filter support.

FIG. 11 schematically illustrates still another variation of the filter of FIG. 3 with a filter region and a counterweight.

FIG. 12 schematically illustrates an example in which the filter of FIG. 2 includes a plurality of the filters of FIG. 3, which can selectively be moved into and out of the X-ray beam path.

FIG. 13 schematically illustrates the example filter of FIG. 12 with one of the plurality of filters in the X-ray beam path.

FIG. 14 schematically illustrates the example filter of FIG. 12 with another one of the plurality of filters in the X-ray beam path.

FIG. 15 schematically illustrates an example in which the filter of FIG. 2 includes a plurality of filter sections with one of the sections in the X-ray beam path.

FIG. 16 schematically illustrates the example filter of FIG. 15 with another one of the plurality of filter sections in the X-ray beam path.

FIG. 17 schematically illustrates another embodiment of the filter of FIG. 2.

FIG. 18 schematically illustrates a perspective view of the filter of FIG. 17.

FIG. 19 schematically illustrates an example of the filter of FIG. 17 in connection with an X-ray tube in the z-y plane of the imaging system.

FIG. 20 schematically illustrates another example of the filter of FIG. 17 in connection with an X-ray tube in the z-y plane of the imaging system.

FIG. 21 schematically illustrates another example of the X-ray sub-system of FIG. 1.

FIG. 22 schematically illustrates an example of a filter of the X-ray sub-system of FIG. 21.

FIG. 23 schematically illustrates an example in which the filter of FIG. 21 includes a plurality of the filters of FIG. 22, which can selectively be moved into and out of the X-ray beam path.

FIG. 24 schematically illustrates an example in which the filter of FIG. 22 includes a plurality of filter sections with one of the sections in the X-ray beam path.

FIG. 25 schematically illustrates the example of the filter of FIG. 24 with another one of the plurality of filter sections in the X-ray beam path.

FIG. 26 schematically illustrates an example of the filter disposed between a source collimator and a bowtie filter.

FIG. 27 schematically illustrates an example of the filter disposed between the X-ray tube and the source collimator.

FIG. 28 schematically illustrates an example of the filter in connection with a detector array with a reference detector(s).

FIG. 29 schematically illustrates an example of the filter in connection with a reference detector(s) disposed between the filter and an examination region.

FIGS. 30 and 31 schematically illustrate an example of the filter of FIG. 17 rotating through the X-ray beam.

FIG. 32 schematically illustrates an example of an angular range of a filter material of the filter of FIG. 17 in connection with an angular range of an integration period.

FIG. 33 illustrates an example method in accordance with an embodiment(s) herein.

FIGS. 34 and 35 schematically illustrate an example which includes an attenuator moveable into the X-ray radiation beam path to completely attenuate the filtered radiation.

### DETAILED DESCRIPTION OF EMBODIMENTS

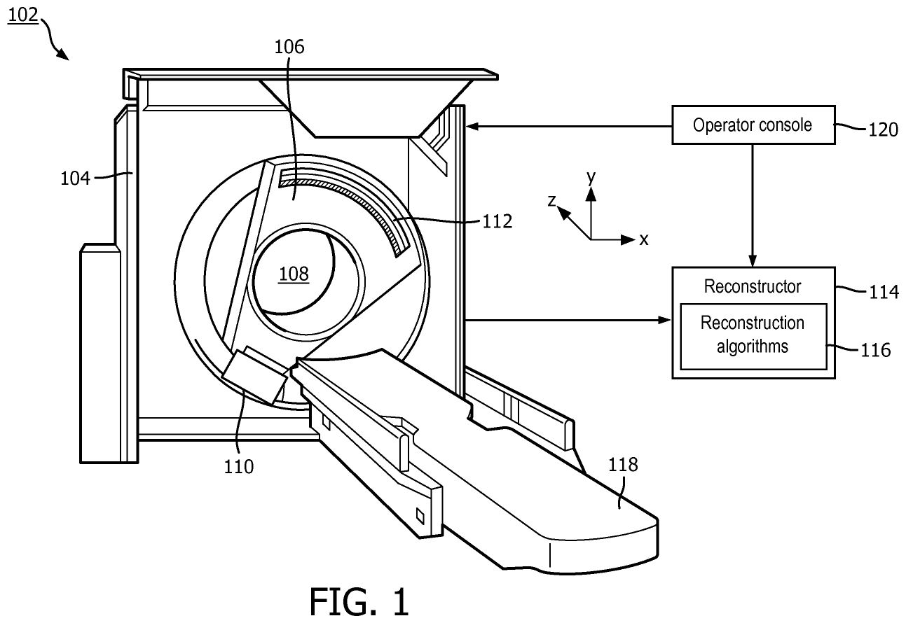

FIG. 1 schematically illustrates an imaging system 100 such as a computed tomography (CT) scanner. The imaging system 102 includes a generally stationary gantry 104 and a rotating gantry 106. The rotating gantry 106 is rotatably supported by the stationary gantry 104 (e.g., via a bearing or the like) and rotates around an aperture 108 (also referred to herein as a bore or an examination region) about a longitudinal or z-axis.

An X-ray sub-system 110 is rotatably supported by the rotating gantry 104, rotates in coordination with the rotating gantry 104, and emits an X-ray radiation. As described in greater detail below, in one instance the X-ray sub-system 110 includes an X-ray source (e.g., an X-ray tube) and a spectral filter, which is configured to selectively filter X-ray photons emitted by the source based on photon energy to produce N different X-ray beams (where N is an integer equal to or greater than two, N≥2), including a first energy spectra X-ray beam, . . . , and an Nth different energy spectra X-ray beam.

The imaging system 100 may also include one or more other X-ray radiation filters. For example, the system 100 may include a beam hardening filter that filters lower energy photons that, in general, will always be absorbed by a scanned subject. Additionally, or alternatively, the system 100 may include a “bowtie” filter to compensate for a shape of the subject to provide a more uniform flux intensity. Additionally, or alternatively, the system 100 may include a source collimator to shape the beam traversing the examination region 108. One or more of these may be integrated with or separate from the X-ray sub-system 110.

A radiation sensitive detector array 112 is rotatably supported by the rotating gantry 104 along an angular arc opposite the X-ray sub-system 110 across the examination region 108. The detector array 112 includes one or more rows of detectors arranged with respect to each other along the z-axis direction. The detector array 112 detects radiation traversing the examination region 108 and generates projection data (line integrals) indicative thereof, including first projection data for detected first energy spectra X-ray photons, . . . , and Nth projection data for detected Nth energy spectra X-ray photons.

A reconstructor 114 reconstructs the projection data with one or more reconstruction algorithms 116. In one instance, the one or more reconstruction algorithms 116 includes one or more spectral reconstruction algorithms and at least one non-spectral reconstruction algorithms. The one or more reconstruction algorithms 116 reconstruct spectral volumetric image data corresponding to one or more different energy spectra. The at least one non-spectral reconstruction algorithm reconstruct non-spectral (e.g., broadband) volumetric image data corresponding to a mean energy spectrum of the X-ray beam.

A subject support 118, such as a couch, supports an object or subject in the examination region 108 so as to guide the subject or object with respect to the examination region 108 for loading, scanning, and/or unloading the subject or object. A computing system serves as an operator console 120, and includes a human readable output device such as a display, an input device such as a keyboard, mouse, and/or the like, one or more processors and computer readable storage medium. Software resident on the console 120 allows an operator to control an operation of the system 100.

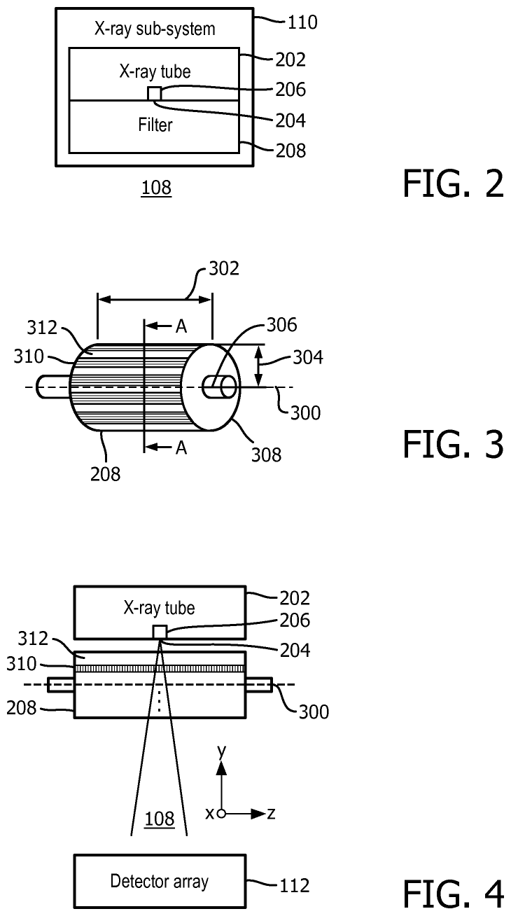

FIG. 2 schematically illustrates an example of the X-ray sub-system 110.

In this example, the X-ray sub-system 110 includes an X-ray tube 202, which includes a focal spot 204 (or a region of an anode of the tube 202 that is bombarded with electrons from a cathode of the tube 202 to produce X-rays) and an X-ray tube port window 206 (which is the exit port for the produced X-rays), and an X-ray energy spectrum (spectral) filter 208. The X-ray energy spectrum filter 208 is spatially located at least in part between the X-ray tube port window 206 and the examination region 108, and filters the X-ray beam energy spectrum prior to the X-ray beam traversing the examination region 108.

FIGS. 3 and 4 schematically illustrates an example of the X-ray energy spectrum filter 208.

In this example, the X-ray energy spectrum filter 208 is cylindrically shaped, having a central axis 300, a height (h) 302, a radius (r) 304 from an origin 306, and a perimeter 308. The X-ray energy spectrum filter 208 includes one or more filter regions 310 of a material(s). Each filter region 310 has a long axis along the height 302, a width along an arc of the perimeter 308, and a depth in a direction radially to the origin 306. In this example, a geometry of each filter region 310 is similar or the same, and the filter regions 310 are arranged parallel to each other around the perimeter 308 and interleaved with spaces 312 in between.

A particular material(s) and/or thickness of each filter region 310 corresponds to a predetermined energy spectrum of interest of the filter 208. For example, in one instance, each filter region 310 is a one millimeter (1 mm±a tolerance) thick filter region 310 of Tin (Sn). The spaces 312 can include another material(s) or be empty. A suitable other material is an X-ray transparent material such as a low-density and low Z material. Another suitable material is a material corresponding to another energy spectrum of interest. The widths of the filter regions 310 and spaces 312 can be equal or not equal, with the widths of the filter regions 310 larger or smaller than those of the spaces 312.

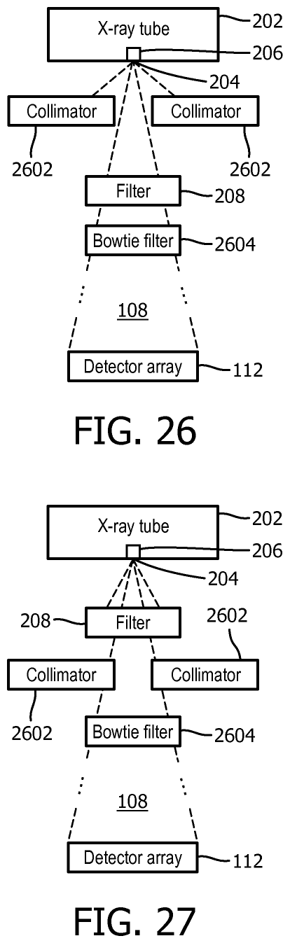

A number and geometry of the filter regions 310 and the spaces 312 in FIG. 3 is for explanatory purposes and is not limiting. FIG. 4 shows a more general case with one or more filter regions 310 and spaces 312, although the geometry of the filter region 310 likewise is for explanatory purposes and is not limiting. The filter 208 can be positioned at a predetermined distance from the port window 206, e.g., as close as possible to the port window 206 or other predetermined distance. (The below description of FIGS. 26 and 27 describes examples of suitable locations). Furthermore, the filter 208 is positioned such that its long axis (the height 302) and the filter regions 310 extend along the z-direction with respect to the X-ray tube 202, as shown in FIG. 4.

The X-ray energy spectrum filter 208 is rotatably supported in this position. A controller, motor, drive system, etc. (not shown) are used to rotate the X-ray energy spectrum filter 208. The X-ray energy spectrum filter 208 rotates about the central axis 300, which is the rotation axis or the axis of rotation. The rotation axis 300 is generally parallel to the z-direction (the axial axis of the imaging scanner 102). As such, the filter regions 310 and the spaces 312 are both parallel to an axial axis of the detectors (the detector slice direction) in the detector array 112.

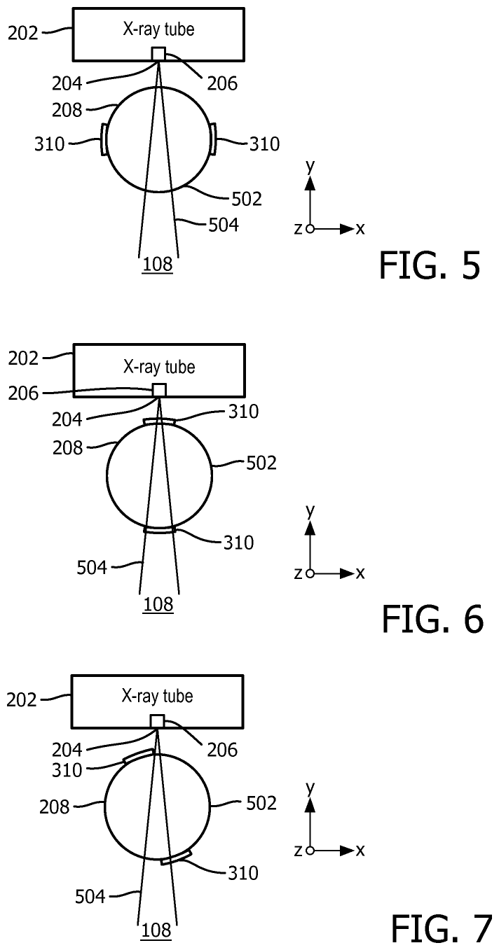

FIGS. 5, 6 and 7 schematically illustrates cross-sectional views of the X-ray energy spectrum filter 208 along a line A-A of FIG. 3, along with the X-ray tube 202, the X-ray port window 206, the focal spot 204, and the examination region 108. FIG. 5 shows the X-ray energy spectrum filter 208 with a pair of filter regions 310 diametrical opposed about a cylindrical support 502 and outside of an X-ray beam 504. FIG. 6 shows the pair of filter regions 310 completely inside of the X-ray beam 504. FIG. 7 shows the pair of filter regions 310 with one of the filter regions 310 partially inside of the X-ray beam 504.

In one instance, each of the filter regions 310 covers a thirty-degree (30 □) arc on the perimeter 308. The remaining three hundred (300 □) includes X-ray transparent or other material, or is an empty space. In other embodiments, the coverage of each filter region 310 can be more or less than thirty-degrees (30 □) and/or there may be more than one pair of filter regions 310. For example, in another instance the X-ray energy spectrum filter 208 includes multiple pairs of filter regions 310 evenly distributed in the circle, each pair covering a smaller angle. An example includes two pairs of filters 90 □ apart on the circle, with each filter region 310 extending 15 □.

When the X-ray energy spectrum filter 208 rotates one rotation, a set of unfiltered data S0 is acquired with the filter regions 310 outside of a path of the beam (FIG. 5), a set of fully filtered data S1 is acquired with both of the filter regions 310 fully in the path (FIG. 6), and a set of partially filtered data S2 is acquired with only one of the filter regions 310 in the path (FIG. 7). The effective acquired data is a weighted sum of S0, S1 and S2. Weights can be pre-computed using the system geometry, including beam fan angle, distance from cylinder axis to the x-ray focal-spot, the cylinder radius, and the width (or angle extended by the thin filter) of the filter region 310 on the cylinder surface. The weights can be calculated for each of the ray tracks as: aS0+bS1+cS2, where a, b, c are the weights for S0, S1, and S2, respectively.

In one instance, the X-ray energy spectrum filter 208 is driven to rotate at a speed that is fast enough to accommodate the highest data acquisition rate the imaging system 102 requires to acquire data to reconstruct images with an unfiltered spectrum. For example, if 1000 data points need to be acquired in a gantry rotation in 0.5 seconds, the X-ray energy spectrum filter 208 is rotated 1,000 times in 0.5 seconds, or 2,000 rps (rotations per second). Motors such as non-contacted motors can reach this speed. For example, small drilling motors can reach 130,000 rpm.

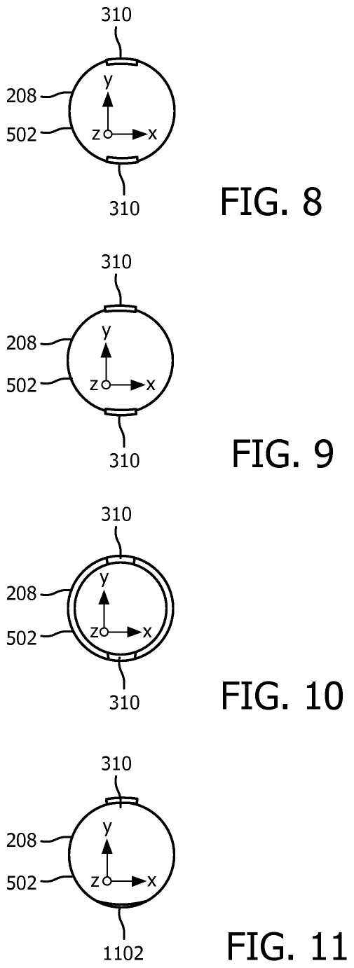

In FIG. 8-11, the filter regions 310 are on an outer surface of the cylinder 502. In FIG. 8, the filter regions 310 are on an inner surface of the cylinder 502. In FIG. 9, the filter regions 310 are on both or span across the inner and the outers surface of the cylinder 502. In FIG. 10, the filter regions 310 are within the cylinder 502. The pairs of diametrically opposed filter regions 310 tend to counter balance each other in FIGS. 8-10. Each of these embodiments could alternatively have a single filter region 310 with another material or volume 1102 of the cylinder 502 opposite thereto that provides a counter balance, e.g., as shown in FIG. 11.

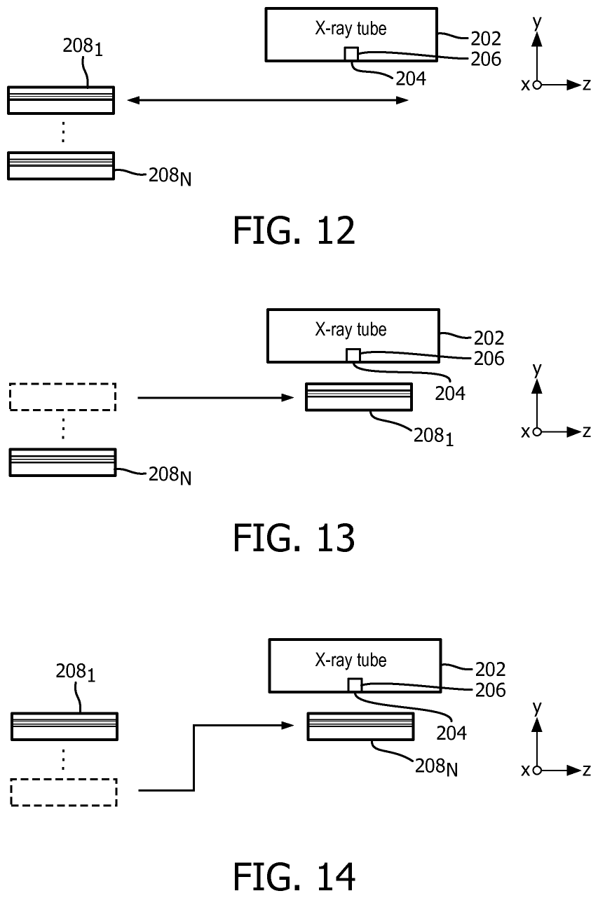

FIGS. 12, 13 and 14 schematically illustrate an example in which the X-ray energy spectrum filter 208 includes a plurality of sub-filters, 208₁, . . . , 208_{N}.

In this configuration, each of the N filters is configured with different filter regions 310 for different spectra filtering. Prior to scanning, a filter of interest is moved into position under the port window 206. The particular sub-filter 208ᵢ may correspond to a selected imaging protocol, anatomy of interest, scan parameter settings (e.g, mAs, kVp, etc.), etc. The sub-filters 208₁, . . . , 208_{N} can be moved via a sub-system that includes a controller, a motor, and drive system. A sub-filter of the filters 208₁, . . . , 208_{N} can be moved in and out of position while the rotating gantry 106 (FIG. 1) is stationary or rotating and/or prior to or during scanning.

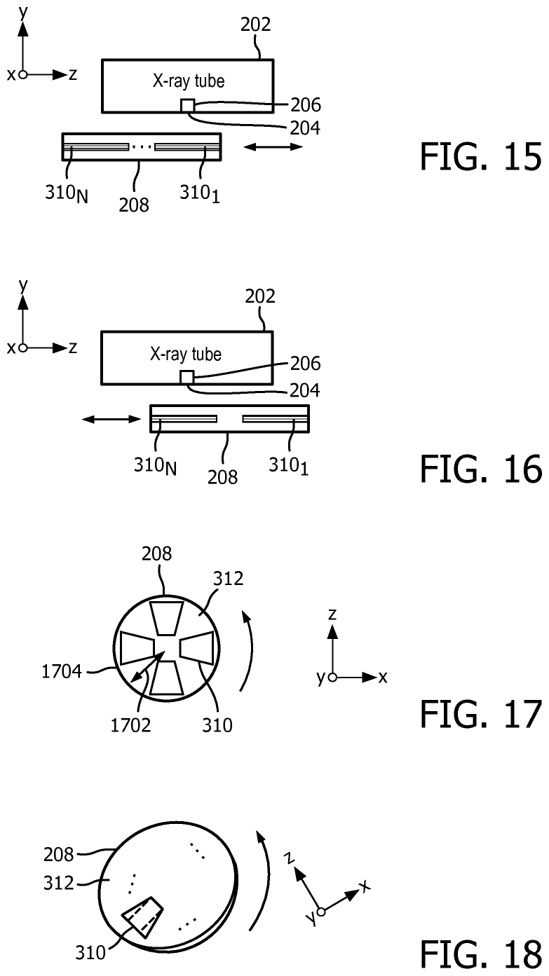

FIGS. 15 and 16 schematically illustrate an example in each of the filter regions 310 includes a row of N filter segments 310₁, . . . , 310_{N}.

In this configuration, each of the N filter segments 310 is configured for different spectra filtering, e.g., via different materials, different volumes of materials, etc. Prior to scanning, a filter segment 310 of interest is moved into position under the port window 206. The particular filter segment 310, may correspond to a selected imaging protocol, anatomy of interest, scan parameter settings (e.g, mAs, kVp, etc.), etc. The X-ray energy spectrum filter 208 can be moved via a sub-system that includes a controller, a motor, and drive system. The X-ray energy spectrum filter 208 can be moved, e.g., translated, in and out while the rotating gantry 106 (FIG. 1) is stationary or rotating and/or prior to or during scanning.

Another variation includes a combination of the examples of FIGS. 12-16, with a plurality of sub-filters, each with a filter region including a row of filter segments.

FIGS. 17 and 18 schematically illustrate a variation of the X-ray energy spectrum filter 208 of FIG. 2.

In this variation, the X-ray energy spectrum filter 208 of FIG. 3 is configured as a circular disc with the filter regions 310 part of a surface thereof. The disc has a radius (r) 1702 and a perimeter 1704. In the example of FIG. 17, four filter regions 310 are evenly distributed on the disc with the spaces 312 therebetween. However, it is to be understood that the disc can include one or more filter regions 310. In this example, each filter region 310 is trapezoid shaped. In another instance, at least one of the filter regions 310 is rectangular shaped, shaped like a slice of pie or pizza, and/or otherwise shaped. FIG. 18 shows the X-ray energy spectrum filter 208 with N filter regions 310.

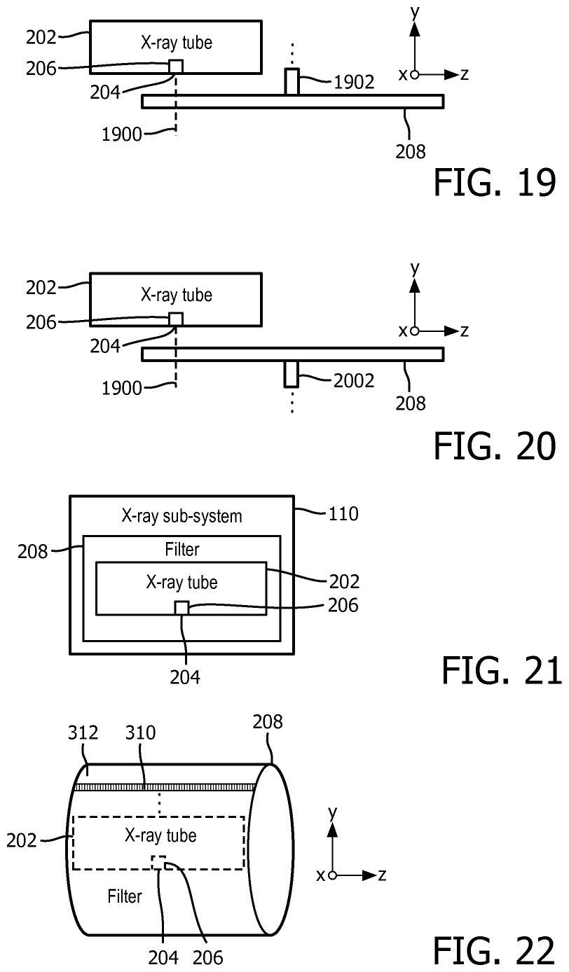

As shown in FIGS. 19 and 20, the X-ray energy spectrum filter 208 is positioned perpendicular to a center 1900 of the x-ray beam. The CT axial axis is in the radial direction of the disc. FIGS. 19 and 20 respectively show different configurations for supporting and/or rotating the filter 208. With FIG. 19, the X-ray energy spectrum filter 208 is supported via a support 1902 that extends towards the X-ray tube 202. With FIG. 20, the X-ray energy spectrum filter 208 is supported via a support 2002 that extends away from the X-ray tube 202. Other configurations are also contemplated herein.

In FIG. 17, for one disc rotation, four data sets from non-filtered spectrum and four data sets from effectively-filtered spectrum are acquired. In this example, where each filter region 310 is trapezoid shaped, when the X-ray energy spectrum filter 208 traverses the X-ray beam, the weight for the filtered spectrum is the same radially, and all the CT slices will have the same effective spectrum. Where each filter region 310 has a rectangular (e.g., square) shape, then different CT slices will see slightly different weights for the filtered spectrum in that a slice farther away from the rotating center of the disc will have a smaller weight from the filtered spectrum than a slice closer to the disc center.

Since the beam is only filtered by one filter region 310, the effective spectrum during a single integration period is cS0+dS1, where the weights c and d are the same for all the ray tracks. When a filter region 310 sweeps in and out of the X-ray beam, there might be an edge effect such as angular scatter, etc. Since the edge sweeps through the imaging field of view uniformly, the average effect is the same across the imaging field of view as seen from the detector array 112 (FIG. 1). In this instance, an iterative reconstruction algorithm can model such effects in the projector/backprojector of the reconstruction algorithm.

FIG. 21 schematically illustrates another variation of the X-ray sub-system 110.

In this variation, the X-ray energy spectrum filter 208 is configured to receive the X-ray tube 202. An example of this is schematically shown in FIG. 22 where the X-ray tube 202 is disposed and enclosed within the cylinder of the filter 208. With this embodiment, the X-ray beam is filtered with only one filter region 310 at a time as the filter region 310 passes over the port window 206.

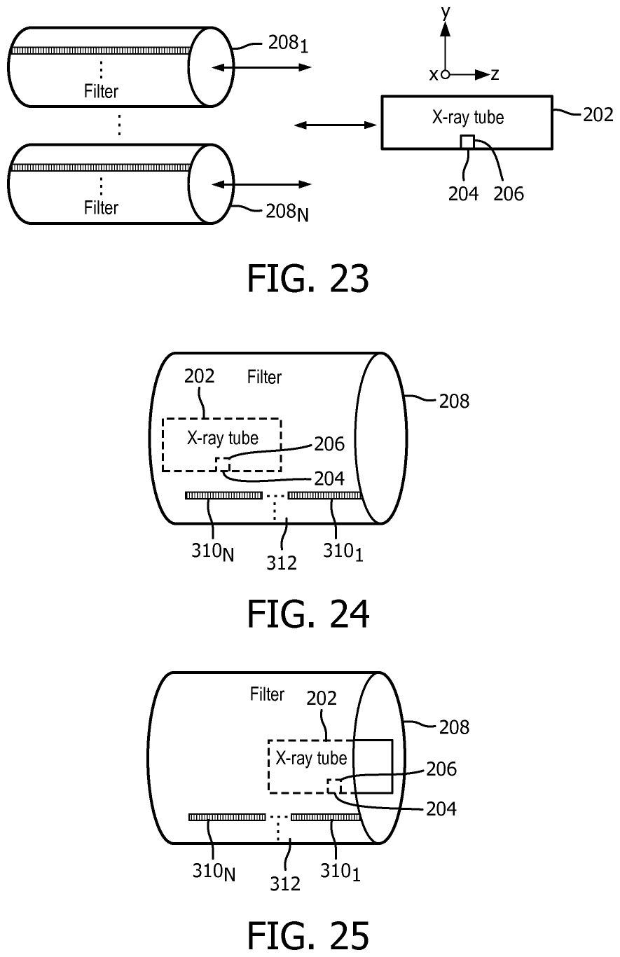

FIG. 23 schematically illustrate an example of the X-ray energy spectrum filter 208 with a plurality of sub-filters 208₁, . . . , 208_{N} of FIG. 22, similar to FIGS. 12-14. FIGS. 24 and 25 schematically illustrate an example of the X-ray energy spectrum filter 208 with a row of filter segments 310₁, . . . , 310_{N} of FIG. 22, similar to FIGS. 15 and 16.

Another variation includes a combination of the examples of FIGS. 23-25, with a plurality of sub-filters, each with a filter region including a row of filter segments.

FIG. 26 schematically illustrates the X-ray energy spectrum filter 208 disposed between a source collimator 2602 and a bowtie filter 2604. In this instance, the beam is first shaped and then filtered. FIG. 27 schematically illustrates the X-ray energy spectrum filter 208 disposed between the X-ray tube 202 and the source collimator 2602. In this instance, the beam is first filtered and then shaped. In either instance, the X-ray energy spectrum filter 208 can be disposed in a filter tray or otherwise so that it can be moved outside X-ray beam path, e.g., for a non-spectral scan. Alternatively, the filter 208 can be held in a static position where the filter region 310 is outside X-ray beam path and not rotated, e.g., for a non-spectral scan.

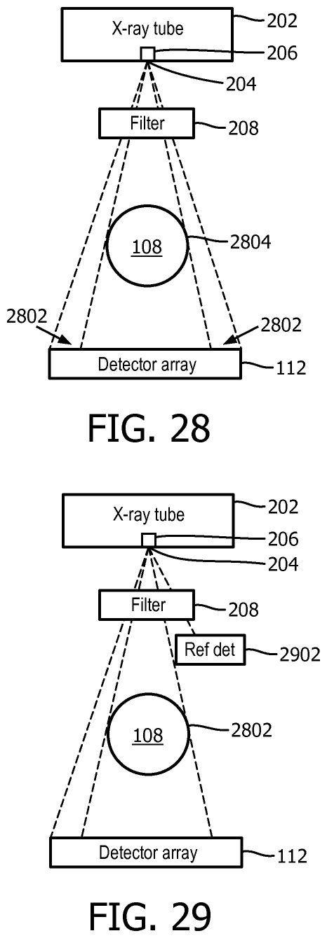

In the embodiments described herein, a rotational position of the X-ray energy spectrum filter 208 can be determined by an X-ray radiation flux detected by a reference detector(s). That is, the X-ray radiation flux will be greatest when there is no filter region 310 in the beam path, smallest when the filter region 310 is completely in the path, and in between and varying (increasing or decreasing) therebetween as the filter region 310 enters the path and leaves the path. In FIG. 28, the reference detector(s) is located in at least one end region(s) 2802 of the detector array 112, outside of a path through a field of view 2804, which is the region in which the subject or object being scanned is positioned for a scan. In FIG. 29, a reference detector(s) 2902 is located between the X-ray energy spectrum filter 208 and the examination region 108, outside of the path through the field of view 2804. In one instance, multiple reference detectors 2902 (e.g., three) can be distributed evenly in an angular range. The positions of the reference detectors 2902 relative to the beam aperture is known. In this case, the rotating speed of the X-ray energy spectrum filter 208 can be detected and used to accurately predict when the X-ray energy spectrum filter 208 will enter or leave the beam aperture.

In one instance, the console 120 starts a timer when an output of the reference detector(s) indicates a filter region 310 is entering the beam path and stops the timer when the output of the reference detector(s) indicates the filter region 310 has left the beam path. The acquired data is synchronized with the start and stop times. As such, the acquired data can be separated into unfiltered and filtered data sets based on the start and stop times. Furthermore, data corresponding to a particular time instance or time interval in the range from the start time to the stop time can be retrieved. As such, the filtered data set can be separated into multiple different acquisition phases. For example, the acquired data can be separated corresponding to the S1 and S2 filtered data.

In another instance, the console 120 uses the detection of the filter region 310 entering the beam path and leaving the beam path to trigger data acquisition. For example, when an output of the reference detector(s) indicates the filter region 310 is in the beam path, data is acquired. When the output of the reference detector(s) indicates the filter region 310 is leaving or has left the beam path, an X-ray attenuating or opaque filter can be moved into the beam path to block the beam from going through the field of view 2804 (as shown in FIGS. 34 and 35 with attenuator 3402), or an electrical current (mAs) of the X-ray tube can be reduced to lower patient dose. Conversely, data is acquired when the filter region 310 is outside of the beam path, and X-rays are blocked or the tube current is reduced when the filter region 310 is fully in the path of the beam.

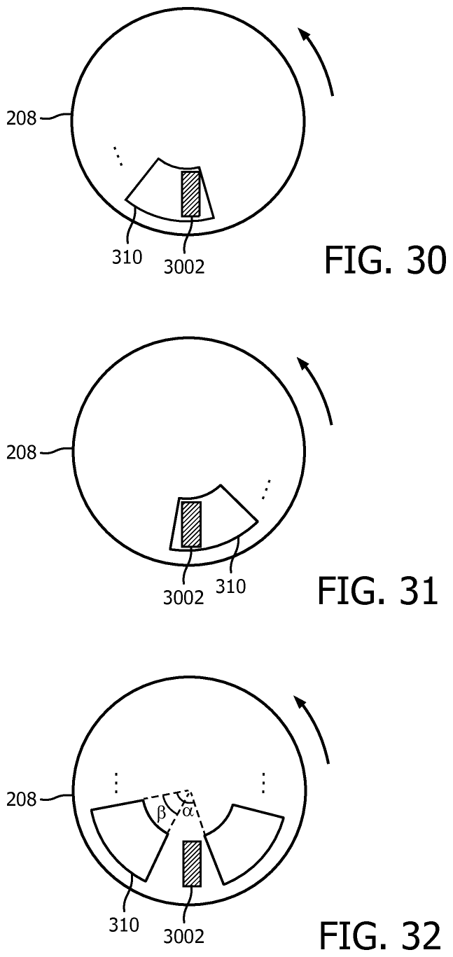

With respect to FIGS. 17-20, 30 and 31, if there is a time gap between two detector integration events (each integration event generates a frame), the position of the X-ray energy spectrum filter 208 and its rotation speed can be configured such that each integration starts after a collimated X-ray beam 3002 already entirely enters the filter region 310 (FIG. 30) and stops just before the collimated X-ray beam 3002 begins to exit the filter region 310 (FIG. 31).

With respect to FIGS. 17-20 and 32, if there is no time gap between two detector integration events, the filter region 310 will enter/exit the X-ray beam 3002 during an integration event. Where the X-ray energy spectrum filter 208 rotates α degrees during the entire integration period and the filter region 310 arc has an angle of β degrees, the filter region 310 begins on one side of X-ray beam 3002 and moves entirely to the other side of X-ray beam 3002. The effective spectrum at the detector 112 during this integration is S_{Eff}=(β/α)S1+((α−β)/α)S0, where S0 and S1 are the original spectrum and spectrum filtered by a filter region 310, and S1(E)=S0(E) e^{μ(E)Δd}, where E is an energy bin, μ(E) is a linear attenuation coefficient of the filter region 310 at energy E, and Δd is a thickness of the filter region 310.

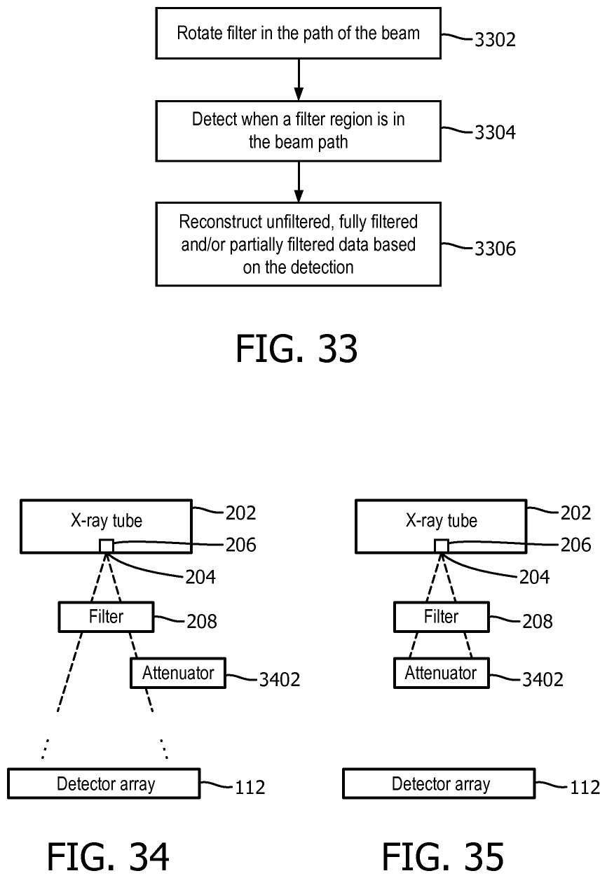

FIG. 33 illustrates an example method in accordance with an embodiment(s) described herein. At 3302, the X-ray energy spectrum filter 208 is rotated in a path of the X-ray beam during a scan. At 3304, the system detects when the filter region 110 is in the path (e.g., partially and/or fully). At 3306, the acquired data is reconstructed to generate volumetric image data based on the detection of the filter region 110. The resulting volumetric image data includes spectral and/or non-spectral volumetric image data, which can be displayed, archived, further processed, etc.

The above may be implemented by way of computer readable instructions, encoded or embedded on computer readable storage medium (which excludes transitory medium), which, when executed by a computer processor(s) (e.g., central processing unit (cpu), microprocessor, etc.), cause the processor(s) to carry out acts described herein. Additionally, or alternatively, at least one of the computer readable instructions is carried by a signal, carrier wave or other transitory medium, which is not computer readable storage medium.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A computed tomography imaging system, comprising:
an X-ray tube having a focal spot and a port window;
a filter having at least a first region with a first material having first X-ray attenuation characteristics for a predetermined X-ray photon energy range of interest, the filter having a second region with a different X-ray attenuation characteristic;
wherein the filter is disposed between the port window and an examination region and is configured to rotate such that the at least first and the second regions sweep through and filter X-ray radiation emitted from the focal spot;
an X-ray radiation flux detector configured to detect an X-ray radiation flux of the filtered X-ray radiation;
a detector array configured to detect the filtered X-ray radiation traversing the examination region and produce a signal indicative thereof; and
a reconstructor configured to process the signal based on the detected flux to reconstruct volumetric image data.

2. The system of claim 1, further comprising:
a processor configured to determine when the first region enters and exits a path of the emitted X-ray radiation.

3. The system of claim 2, wherein the processor is further configured to synchronize the signal with an entry time the first region enters the path and an exit time the first region exists the path.

4. The system of claim 3, wherein the reconstructor is configured to process only a sub-portion of the signal corresponding to the entry and exit times.

5. The system of claim 3, wherein the processor determines a filter time as a time period from the entry time to the exit time, and the reconstructor is configured to process only a sub-portion of the signal corresponding to a sub-time range of the filter time which is less than the filter time.

6. The system of claim 3, wherein the processor determines a filter time as a time period from the entry time to the exit time, and the reconstructor is configured to process a plurality of different sub-portions of the signal corresponding to a plurality of sub-time ranges of the filter time, each of the plurality of sub-time ranges corresponding to a different phase.

7. The system of claim 3, wherein the processor is further configured to trigger data acquisition based on the entry and exit times.

8. The system of claim 7, wherein the processor is further configured to move an X-ray attenuating filter into the path to attenuate the emitted X-ray radiation outside of the filter time.

9. The system of claim 7, wherein the processor is further configured to reduce a tube electrical current of the X-ray tube outside of the filter time.

10. The system of claim 1, wherein the filter is cylindrically shaped and configured to surround the X-ray tube.

11. The system of claim 10, wherein the filter includes a plurality of cylindrically shaped filters, each of the cylindrically shaped filters is configured to surround the X-ray tube, and only a single one of the cylindrically shaped filters is positioned to surround the X-ray tube at a given time.

12. The system of claim 10, wherein the first region includes at least two segments, each segment having a different X-ray attenuation characteristic, and the filter is configured to translate to alternately position only a single one of the segments in front of the port window.

13. The system of claim 1, wherein the filter includes a plurality of filters, each of the filters is configured to move entirely between the port window and the examiner region, and only a single one of the filters is positioned in front of the port window at a given time.

14. The system of claim 1, wherein the first region includes at least two segments, each having a different X-ray attenuation characteristic, and the filter is configured to translate to alternately position only a single one of the segments in front of the port window.

15. A method, comprising:
rotating a filter in a path of X-ray radiation emitted form an X-ray tube of an imaging system during scanning, wherein the filter includes at least a first region with a first material having first X-ray attenuation characteristics for a predetermined X-ray photon energy range of interest, wherein the filter includes a second region with a different X-ray attenuation characteristic;
detecting a position of the filter based on an X-ray radiation flux; and
processing acquired data based on the detected flux to reconstruct volumetric image data of interest.

16. The method of claim 15, further comprising:
extracting a sub-portion of the acquired data corresponding to a time period only in which the first region sweeps through the path; and
reconstructing only the sub-portion to reconstruct spectral volumetric image data.

17. The method of claim 15, further comprising:
extracting a sub-portion of the acquired data corresponding to a time period only in which the first region is outside of the path; and
reconstructing only the sub-portion to reconstruct non-spectral volumetric image data.

18. A computed tomography imaging system, comprising:
an X-ray tube;
a filter with at least a first region with a first material having first X-ray attenuation characteristics for a predetermined X-ray photon energy range of interest, the filter having a second region with a different X-ray attenuation characteristic;
wherein filter is cylindrically shaped, configured to surround the X-ray tube, and configured to rotate such that the at least first and the second regions sweep through and filter X-ray radiation emitted from the X-ray tube;
a detector array configured to detect the X-ray radiation traversing the filter and produce a signal indicative thereof; and
a reconstructor configured to process the signal to reconstruct volumetric image data.

19. The system of claim 18, further comprising an X-ray radiation flux detector configured to detect an X-ray radiation flux of the filtered X-ray radiation, wherein the reconstructor is configured to process the signal based on the detected X-ray radiation flux.

20. The system of claim 18, wherein filter includes a plurality of cylindrically shaped filters, and further comprising a drive system configured to move a predetermined one of the plurality of cylindrically shaped filters over the X-ray tube and into a path of the emitted X-ray radiation.

21-25. (canceled)
